(19) United States
(12) Patent Application Publication
(10) Pub. No.: US 2025/0147001 A1
(43) Pub. Date: May 8, 2025

(54) METHOD FOR DETERMINING A PARAMETER OF A COMPOSITION OF NATURAL ORIGIN FOR DETERMINING THE MOST SUITABLE TREATMENT FOR THE REMOVAL OF HETEROATOMS FROM SAID COMPOSITION

(71) Applicant: TotalEnergies OneTech, COURBEVOIE (FR)

(72) Inventors: Alice LIMOGES, Solaize (FR); Ségolène LAAGE, Solaize (FR); Florent PICARD, Solaize (FR); Christine LACOUX, Solaize (FR)

(73) Assignee: TotalEnergies OneTech, COURBEVOIE (FR)

(21) Appl. No.: 18/835,956

(22) PCT Filed: Feb. 13, 2023
(86) PCT No.: PCT/FR2023/050186

§ 371 Date: Aug. 5, 2024

(30) Foreign Application Priority Data
Feb. 15, 2022 (FR) FR2201327

(51) Int. Cl. G01N 33/26; C11B 3/00; C11C 3/12

(52) U.S. Cl. CPC G01N 33/26; C11B 3/006; C11C 3/12

(57) ABSTRACT

The present invention concerns a method for determining a parameter representative of the quality of a composition of natural origin, this parameter making it possible to determine the most appropriate heteroatom removal treatment for processing this composition, in particular with a view to its hydrotreatment for the manufacture of renewable fuels. The invention also concerns a process for reducing the heteroatom content of this type of composition, in particular for manufacturing a renewable fuel.

### FIELD OF THE INVENTION

The present invention relates to a method for determining a parameter representative of the quality of a composition of natural origin, said parameter making it possible to determine the most suitable treatment for the removal of heteroatoms for treating said composition, in particular with a view to the treatment thereof for producing renewable fuels, in particular of the diesel and/or aircraft fuel type (in particular SAF: “Sustainable Aviation Fuel). The invention also relates to a chaining of methods for processing said type of composition, in particular for producing a renewable fuel.

### PRIOR ART

Different types of renewable fuels are available today: biodiesel, and fuel coming from the hydrotreatment of oils of natural origin.

Biodiesel is currently produced by transesterification of triglycerides with methanol, producing methyl ester and glycerol in the presence of a homogenous or heterogenous basic catalyst. Triglycerides come from oils of natural origin.

Other renewable fuels are produced by hydrodeoxygenation of oils of natural origin, generally followed by a step of isomerization making it possible to improve the cold properties of the fuel, and optionally a step of fractionating. The steps of hydrodeoxygenation and isomerization are also carried out in the presence of a catalyst.

The oils of natural origin usually used to manufacture renewable fuels, are animal fats and oils, various seed oils, soy, rapeseed, palm, seed oils that were collected after having been used to prepare food, commonly called used cooking oils (UCO), or algal oils or oils coming from the shells of nuts, in particular cashew nut shell oils.

These oils and fats can however contain high contents in pollutants (phospholipids, phosphate salts, gums, metals, sulfur, ash, water, pigments and other undesirable materials) able to have a deleterious effect on later treatments, with for example deactivation of the catalysts of the methods downstream, corrosion, fouling, etc. It is therefore necessary to pretreat them in order to remove all or part of these harmful effects.

As such, before they are converted into renewable fuel, fats and oils of natural origin and UCOs are usually pretreated by well-known chemical and physical methods, similar to those implemented for the treatment of food oils, such as degumming, neutralization with an alkaline solution (generally NaOH) or acid solution (for example citric acid) bleaching, finishing or polishing, steam treatment, etc.

Document US2019338219A1 thus describes a method for producing biodiesel comprising a step of pre-treatment before esterification. The pre-treatment is chosen according to the fatty acid content of the feedstock. This can be a chemical refining with the adding of a basic compound followed by centrifugation then bleaching and polishing, or degumming with the adding of a basic compound also followed by centrifugation then bleaching and polishing, or only bleaching and polishing. The step of centrifugation makes it possible to remove the aqueous phase, polar or hydratable compounds and solids. The step of bleaching (by heating or adding a bleaching agent) makes it possible to remove the solids, residual soaps, humidity and other impurities. The step of polishing makes it possible to remove residual solids by filtration. If fatty acids still remain, they can be removed either by stripping (or deacidification) or converted by esterification before the transesterification reaction. The biodiesel produced is then again subjected to purification by filtration.

A method for purification is also known from document US2019031964A1 that makes it possible to notably reduce the sulfur, metals and other impurities in renewable oils. The oil is first mixed with an aqueous solution of citric acid at 60° C., then subjected to centrifugation in order to remove the gums and finally treated with a mixture containing water, a hydrotreated long-chain ester compound and a phosphate derivative at at least 100° C. for 10 minutes, then the temperature is increased to 120° C.-130° C. for 30 to 90 minutes. The mixture is then cooled and again centrifuged.

However, these well-known methods consume chemicals, generate waste and can consume much energy, in particular when it is necessary to heat. Furthermore, due to the complexity of oils, removing all over the gum products can be difficult, and this all the more so that, most of the time, mixtures of oils and fats of different origins are used and the relative ratios thereof can fluctuate substantially over time.

It is therefore necessary to have a flexible and robust method to correctly remove the phosphatides, nitrogen compounds and metals of complex mixtures of oils and fats of different origins and of fluctuating composition.

Removing phosphatides, as well as nitrogen compounds, via the usual pre-treatment methods remains difficult however and hardly predictable so that only complex mixtures of oils and fats that have specific phosphorus contents are today accepted at the input of these pre-treatments, which limits the choice of potentially usable feedstocks. Thus, currently, the treated mixtures have a total elemental phosphorus content less than or equal to a specific threshold. Some of these feedstocks however remain difficult to treat even when they have an elemental phosphorus content less than this threshold.

There is therefore a need to more efficiently treat and at a lesser cost compositions of natural origin comprising lipids and heteroatoms, in particular at least one heteroatom chosen from nitrogen and phosphorus. These heteroatoms are typically in the form of organic compounds.

### SUMMARY

The applicant has discovered that, surprisingly, the ratio of the total heteroatom content present in pH neutral hydratable compounds over the total content in this heteroatom, in particular when the heteroatom is phosphorus or nitrogen, makes it possible to characterize the quality of a composition of natural origin containing lipids, and in particular its aptitude to be treated efficiently via a heteroatom removal treatment.

The object of this invention is to propose a method for determining a parameter representative of the quality of a composition of natural origin containing lipids, making it possible to determine its aptitude to be treated by a treatment for the removal of heteroatoms in order to reach a target heteroatom content or less than a target value efficiently and at less cost.

This parameter can be used to determine which heteroatom removal treatment can be applied to a composition of oil of natural origin containing lipids to reach a target heteroatom content, in particular in phosphorus and/or nitrogen.

The parameter can also be used in a method for decreasing the heteroatom content of a composition of natural origin containing lipids, in particular a method for decreasing the heteroatom content.

### DETAILED DESCRIPTION

### Method for Determining a Parameter Representative of the Quality of a Composition of Natural Origin

The first object of the invention relates to a method for determining a parameter representative of the quality of a composition of natural origin comprising heteroatoms and lipids chosen from phenolic lipids, fatty acids, triglycerides, diglycerides, monoglycerides, phospholipids, fatty acid esters and/or a mixture of two or more of these compounds, the method comprising:
(A) determining the total content of said composition in at least one heteroatom chosen from phosphorus and nitrogen,
(B) identifying compounds of the at least one heteroatom which are hydratable at neutral pH and determining the total content of said composition in the at least one heteroatom present in the identified neutral pH hydratable compounds of this heteroatom,
(C) determining as a parameter representative of the quality of the composition of the ratio of the total content in the at least one heteroatom present in the identified neutral pH hydratable compounds of this heteroatom over the total content of the composition in this heteroatom, this ratio being determined for phosphorus, for nitrogen or for each one of these two heteroatoms.

In particular, the step (B) can be carried out by means of an analysis technique chosen from NMR, mass spectroscopy, ion chromatography, liquid chromatography coupled with mass spectrometry (LC-MS), high-performance thin-layer chromatography (HPTLC).

Phosphorus 31 (noted as ³¹P) or nitrogen 15 (noted as ¹⁵N) NMR can thus be used.

The determination carried out in step (A) can be carried out by an elemental analysis, typically by X-ray fluorescence (XRF) or by ICP (in particular according to standard UOP 389), although other methods can be considered (NMR, HPTLC, etc.).

The total contents can be contents by weight, for example expressed in ppm, or concentrations, for example a molar concentration or other.

The aforementioned analysis techniques are well known to those skilled in the art who will know how to implement them, and in particular prepare the samples, to identify the neutral pH hydratable compounds and quantify the phosphorus and/or nitrogen present in this composition.

For example, for ³¹P NMR, the sample can undergo a washing with a chelating agent (for example of the EDTA type or other) with a suitable pH (for example a pH=7) then the organic and aqueous phases are separated and the analysis by ³¹P NMR is then carried out on each one of the recovered phases.

Typically, the neutral pH hydratable nitrogen or phosphorus compounds that are to be identified and for which the P and/or N content is to be qualified are compounds for which the structure and potential presence are known, for example following a prior analysis of the composition.

### Composition of Natural Origin

The composition of natural origin used in the present invention is a composition comprising heteroatoms and lipids chosen from phenolic lipids, fatty acids, triglycerides, diglycerides, monoglycerides, phospholipids, fatty acid esters and/or a mixture of two or more of these compounds.

This composition can comprise, or consist of, an oil of natural origin or a mixture of natural oils.

An oil of natural origin is defined as an oil that does not contain any mineral oil of fossil origin.

The composition according to the invention can contain one or more oils of natural origin chosen from a vegetable oil, an animal oil or fat, a used oil, an oil produced by microorganisms, esters resulting from the transesterification of fatty acid esters contained in one or more of these oils, as well as mixtures thereof.

Typically, an oil of natural origin can contain 50% m or more, 60% m or more, preferably 70% m or more, of phenolic lipids, fatty acids and/or fatty acid esters (mono-, di-, triglycerides, fatty acid ethyl esters, fatty acid methyl esters).

An oil of natural origin can contain 50% m or more of fatty acid esters (mono-, di-, triglycerides, fatty acid ethyl esters, fatty acid methyl esters) and/or fatty acids, preferably 60% m or more, preferably 70% m or more.

In one embodiment, an oil of natural origin or a mixture of oils of natural origin, can contain fatty acid esters and free fatty acids, containing one to three saturated or unsaturated C8-C24 acyl groups. When several acyl groups are present, they can be identical or different.

Compositions resulting from the transesterification of the fatty acid esters contained in these oils, such as compositions comprising fatty acid methyl esters or fatty acid ethyl esters, and comprising impurities coming from the oils, can also be part of the treated compositions of natural origin considered in the present invention.

An oil of natural origin can contain 50% m or more, preferably 60% m or more, preferably 70% m or more, of phenolic lipids. These phenolic lipids comprise in particular the compounds represented by formula (1):

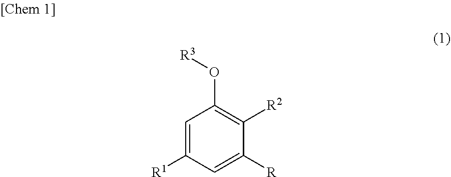

Where:
R is a C10-C30 straight chain alkyl group, optionally C12-C20, for example in C15, saturated or unsaturated, substituted or not by heteroatoms chosen from O, N or S,
R¹ is hydrogen or a hydroxyl group,
R² is hydrogen, a carboxylic group or an ester,
R³ is hydrogen.

The oil of natural origin can in particular comprise one or more of the following phenolic lipids:
alkylated phenols of which the alkyl group is a C10-C30 straight chain, optionally C12-C20, for example in C15, saturated or unsaturated, substituted or not by heteroatoms chosen from O, N or S,
alkylresorcinols of which the alkyl group is a C10-C30 straight chain, optionally C12-C20, for example in C15, saturated or unsaturated, substituted or not by heteroatoms chosen from O, N or S,
anacardic acids, of which the alkyl group is a C10-C30 straight chain, optionally C12-C20, for example in C15, saturated or unsaturated, substituted or not by heteroatoms chosen from O, N or S.

The vegetable oil can be chosen from pine oil, rapeseed oil, sunflower oil, castor oil, peanut oil, linseed oil, babassu, hemp oil, linola oil, jatropha oil, peanut oil, rice bran oil, mustard oil, carinata oil, coconut oil, copra oil, olive oil, palm oil, cotton oil, corn oil, palm kernel oil, soybean oil, marrow oil, grape seed oil, argan oil, jojoba oil, sesame oil, walnut oil, hazelnut oil, tung oil, rice oil, safflower oil, algal oil, used oils, nut shell oil (in particular cashew nut shell oil), and any combination thereof.

Used oil includes used cooking oils (used food oils) and the oils recovered from residual water, such as fats/oils from traps and drains, gutter oils, sewage oils, for example from water treatment plants, and the used fats from the food industry.

The animal fat can be chosen from tallow, lard, fat (yellow and brown fat), fish oil/fat, milk fat.

The oil of natural origin can also be an oil produced by natural or genetically modified microorganisms, such as bacteria, yeasts, in particular oleaginous yeasts, algae, prokaryotes or eukaryotes. In particular, these oils can be recovered by well-known mechanical or chemical extraction methods.

The aforementioned oils of natural origin, of which most are rich in triglycerides or in phenolic lipids, furthermore contain components such as free fatty acids, mono and di-glycerides, and/or many other organic and inorganic components, in particular phosphatides, sterols, tocopherols, tocotrienols, hydrocarbons, pigments (gossypol, chlorophyll), vitamins (carotenoids), sterol glycosides, glycolipids, protein fragments, traces of pesticides and traces of metals, as well as resinous and mucilaginous materials. Among the latter components, certain compounds, in particularly those that contain heteroatoms, are pollutants that should preferably be eliminated at least partially before a later treatment.

The renewable oil composition according to the invention thus typically includes heteroatoms including in particular phosphorus and/or nitrogen. These heteroatoms are generally in the form of organic compounds, in particular in the form of lipids.

The phosphorus content of the composition of natural origin can be 20 ppm or more or 50 ppm or more, for example from 50 ppm to 1500 ppm, or from 200 ppm to 1200 ppm, measured for example by X-ray fluorescence or ICP by the method UOP 389.

The nitrogen content of the composition of natural origin can be 50 ppm or more, for example from 50 ppm to 1200 ppm or from 200 ppm to 2000 ppm, measured for example by X-ray fluorescence.

The composition of natural origin can furthermore comprise one or more other heteroatoms such as alkaline metals, in particular potassium, alkaline earth metals, and or chlorine. The content in these heteroatoms can be variable according to the constituents of the composition. It can be determined by an elemental analysis of the X-ray fluorescence type or by ICP.

In the composition of natural origin considered in the invention, phosphorus can in particular be present in the form of phosphatides, of which the most common are phosphatidic acid, phosphatidylethanolamine, phosphatidylcholine, phosphatidylinositol and phosphate salts. As these compounds are often charged due to their low (phosphate group) or high (amino group) pKa, they can also contain alkaline or alkaline earth elements or absorb metal cations such as copper or iron.

FIG. 3 shows the various forms of these phospholipids in water according to the pH, the R group represents a diglyceride. Note as such that the phosphatidylcholine (noted as PC) and the phosphatidylinositol (noted as PI) are hydratable regardless of the pH, that the phosphatidylethanolamine (noted as PE) is hydratable to at pH 2-3, while phosphatidic acid (noted as PA) is hydratable for pH greater than 4 if it is not chelated by cations present in the mixture, in which case it will remain in the organic phase.

The term “hydratable compound” means a compound that is at least partially soluble in water. This solubility can depend on the pH, as explained hereinabove.

In the present invention, the following shall be considered as neutral pH hydratable compounds (pH=7): compounds at least partially soluble or fully soluble in neutral pH water, in particular in the temperature conditions of the treatment implemented. A component is considered to be at least partially soluble in neutral pH water when at least 40% m of this component is solubilized, in particular in the temperature conditions of the treatment implemented. The conditions of the treatment implemented are in particular those of the washing with neutral pH water described hereinafter.

The neutral pH hydratable phosphorus compounds are in particular hydratable phosphate salts, phosphatidylcholine and phosphatidylinositol, and, to a lesser degree, phosphatidylglycerol.

Nitrogen is present in phosphatidylcholine and phosphatidylethanolamine. It can also be present in the composition of natural origin in the form of chlorophyll, protein residues, fatty amines, etc.

The neutral pH hydratable nitrogen compounds are in particular phosphatidylcholine and, to a lesser degree, phosphatidylinositol.

The invention is not however limited to these compounds, and all identifiable neutral pH hydratable compounds can in particular be taken into account.

In particular, in order to identify and quantify neutral pH hydratable compounds, an analysis can be conducted on the composition to be treated so as to identify the organic compounds containing phosphorus and/or nitrogen, isolate these compounds and proceed with solubility tests in water, in particular at different pH levels in temperature conditions of the treatment implemented. The identification of the compounds can in particular be carried out using the techniques mentioned hereinabove (NMR, mass spectroscopy, ion chromatography, liquid chromatography coupled with mass spectrometry and high-performance thin-layer chromatography).

### Method for Determining the Treatment for the Removal

Another object of the invention relates to a method for determining the treatment for the removal of heteroatoms to be applied to a composition of oil of natural origin, comprising heteroatoms and lipids chosen from phenolic lipids, fatty acids, triglycerides, diglycerides, monoglycerides, phospholipids, fatty acid esters and/or a mixture of two or more of these compounds, to reach a target content heteroatom, in particular in phosphorus and/or nitrogen.

The method according to the invention comprises:
(a) a step of determining at least one parameter representative of the quality of the composition using the method for determining according to the invention,
(b) a step of estimating a residual content of at least one heteroatom chosen from among phosphorus and nitrogen, using the representative parameter, this residual content corresponding to the content of at least one heteroatom after removal of the hydratable compounds at pH=7 containing this heteroatom,
(c) a step of determining a treatment to be applied to said composition to reach a target content in at least one heteroatom, in particular chosen from phosphorus and/or nitrogen, at the output of the treatment, said treatment being chosen from
   (i) a washing with neutral pH water,
   (ii) a washing with neutral pH water followed by a step of removing non-hydratable compounds,
   (iii) a washing with neutral pH water followed by a step of removing compounds hydratable at a pH other than a neutral pH,
   (iv) a washing with neutral pH water followed by a step of removing compounds hydratable at a pH other than a neutral pH then a step of removing non-hydratable compounds,
wherein
said residual content is compared to the target content and

The treatment (i) is chosen if the residual content is less than or equal to the target content,
otherwise, a database or a model is used configured to supply, an estimate of the content in the at least one heteroatom at the output of each treatment (ii) to (iv) according to a content in this heteroatom at the input of the treatment equal to the residual content, and the treatment is chosen for which the estimated content at the output is less than or equal to the target content, and optionally for which the number of treatment steps is the lowest.

It is thus possible to determine the treatment, in particular the treatment having the least steps, making it possible to obtain a target heteroatom content, in particular in P and/or N. The treatment thus makes it possible to reach this target content regardless of the initial phosphorus and/or nitrogen content of the composition.

A treatment making it possible to reach a target content either for phosphorus, or for nitrogen, or for both, can in particular be chosen. A determination is then made in step (a) of the parameter representative of the quality of the composition for the phosphorus, the parameter representative of the quality of the composition for the nitrogen, or both parameters, and the database or the model used in step (c) are then configured to supply an estimate of a phosphorus content, of a nitrogen content, or both, at the output of each treatment (ii) to (iv) according to a content in this heteroatom at the input of the treatment equal to the residual content.

It may occur that, during step (c), none of the estimated contents at the output are less than or equal to the target content. In this case, steps (a) to (c) can advantageously be reiterated by diluting said composition of natural origin with another composition of natural origin, in particular a composition having a parameter representative of its quality greater than that of said composition (for example determined beforehand by the method for determining according to the invention).

This can make it possible to use a feedstock for which the usual treatments for the removal of heteroatoms are not sufficiently effective to sufficiently reduce the quantity of heteroatoms, in particular phosphorus and/or nitrogen, to reach a target specification.

Advantageously, the database and/or the model can be configured to supply an estimate of the contents of at least one heteroatom at the output of said treatment obtained for different operating conditions of each treatment (i) to (iv) and during the step (c), the treatment and the operating conditions for this treatment can then be chosen for which the estimated content at the output is less than or equal to the target content, and optionally for which the number of treatment steps is the lowest and/or for which the quantity of reagent used is the lowest. It is thus possible to optimize the treatment conditions for each feedstock, which makes it possible in particular to reduce the quantities of reagents used, and therefore the costs.

The method for determining according to the invention can in particular be used for a composition of natural origin such as defined hereinabove.

The database or the model used in the present method are as such configured to supply estimates of heteroatom contents (at least in P and/or N) at the output of a treatment. They can be established beforehand by means of tests during which compositions of natural origin are subjected to each one of the treatments (i) to (iv). It is thus possible to measure for each test and each type of treatment, optionally for different operating conditions of each treatment:
the total content of each composition in at least one heteroatom chosen from phosphorus and nitrogen, before and after treatment,
the total content of each composition in at least one heteroatom contained in the neutral pH hydratable compounds of this heteroatom.

The procedure for determining according to the invention can further comprise a step (d) of treating said composition, wherein the latter is subjected to the treatment chosen in the step (c) and a composition of natural origin is recovered having a reduced content in at least one heteroatom, in particular in at least one heteroatom chosen from phosphorus and nitrogen, and advantageously in at least one other heteroatom, in particular chosen from metals, alkaline metals, earth alkalines and chlorine.

The composition thus treated can then be subjected to a step of hydrotreatment, optionally in mixture with hydrocarbons of fossil origin, then advantageously to a step of isomerization, so as to produce a renewable fuel, in particular of the diesel type.

Alternatively, the composition thus treated can be subjected to a step of transesterification or esterification to produce a fuel of the biodiesel type.

The steps for the treatments for the removal (i) to (iv) as well as the step of hydrotreatment can be such as described hereinbelow in reference to the method for decreasing the content in heteroatoms of a composition of natural origin according to the invention.

### Method for Decreasing the Heteroatom Content

The invention also has for object a method for decreasing the heteroatom content of a composition of natural origin comprising heteroatoms and lipids chosen from phenolic lipids, fatty acids, triglycerides, diglycerides, monoglycerides, phospholipids, fatty acid esters and/or a mixture of two or more of these compounds, in particular such as defined herein above.

In a first embodiment, the method comprises:
(E1) a step of determining the treatment for the removal of heteroatoms to be applied to the composition of natural origin to reach a target content in at least one heteroatom, by means of the method for determining according to the invention, and
(E2) a step during which said composition of natural origin is subjected to the treatment determined in step (E1).

In a second embodiment, the method comprises:
(E′2) A step for the removal of heteroatoms during which the composition of natural origin is subjected to a treatment chosen from:
   (i) a washing with neutral pH water,
   (ii) a washing with neutral pH water followed by a step of removing non-hydratable compounds,
   (iii) a washing with neutral pH water followed by a step of removing compounds hydratable at a pH other than a neutral pH,
   (iv) a washing with neutral pH water followed by a step of removing compounds hydratable at a pH other than a neutral pH then a step of removing non-hydratable compounds,
and wherein, the composition of natural origin has a parameter representative of the quality of the composition, determined beforehand using the method for determining according to the invention, which is greater than or equal to a threshold beyond which the effectiveness of the step of removing is sufficient to reduce the heteroatom content of said composition to a target content or less than a target content.

This makes it possible to select, upstream of the step for removing, a composition of natural origin for which a particular treatment makes it possible to obtain a particular specification. This can in particular make it possible to optimize the operation of an existing treatment unit.

These threshold values of the parameter can be determined beforehand using a database identical to that described herein above, for example by a statistical treatment of data of the base and/or by means of a model such as described herein above.

Each one of the four treatments (i) to (iv) that can be considered comprise a step of washing with neutral pH water, which can be followed, according to the treatment, by a step of removing compounds hydratable at a pH other than a neutral pH. Each one of these steps is detailed hereinafter.

In each one of the embodiments, the method for decreasing can further comprise a step of hydrotreatment (E3) of the product directly coming from the step (E2) or (E′2).

Step of Washing with Neutral pH Water

Washing said composition of natural origin with neutral pH water makes it possible to notably decrease the heteroatom content, in particular in heteroatoms chosen from phosphorus, nitrogen, chlorine, alkaline metals, alkaline earths, in particular it makes it possible to remove the compounds of nitrogen and/or phosphorus contained in the neutral pH hydratable compounds of these atoms.

This step of washing with water is carried out with a neutral pH water, in other words without adding a chemical (acid or base) aimed at modifying the pH. This is typically washing with demineralized water. This washing is generally carried out with stirring in order to favor the contact between the water and the oil.

When the residual heteroatom content (in particular P and/or N) estimated from the representative parameter is less than the target content, this washing can be sufficient to remove all, or almost all, of these heteroatoms without having to carry out other treatments for removing heteroatoms such as the additional steps for removing provided for in treatments (ii) to (iv).

Typically, this washing with water is carried out at atmospheric pressure, at a temperature ranging from ambient temperature to a temperature at which the composition is liquid. This temperature can be determined according to the pour point of the composition. It can be greater than 50° C., for example from 50° C. to 100° C.

This step of washing is typically carried out by putting the composition into contact with the neutral pH water followed by a separating of the oily and aqueous phases, the recovered oily phase forming a composition having a reduced content in neutral pH hydratable compounds. This putting into contact can be reiterated one or more times. After each putting into contact, the oily phase and the aqueous phase are then separated, and the separated oily phase is put into contact again with neutral pH water or recovered. This separation can be carried out by means of any usual separating technique.

This washing can in particular be carried out by putting the composition into contact with neutral pH water, preferably under stirring, for a duration that is sufficient to mix the two phases, for example for a duration of 15 min to 1 h.

Advantageously, during the step of washing, the water/composition mass ratio can be 1:99 to 10:90. Small quantities of water are sufficient indeed to notably lower the heteroatom content.

This step of washing can be carried out in a specific chamber, dedicated to this step. A chamber can for example be used that has a water/oil mixing device (recirculation pump with water injection upstream of the pump, stirring device, etc.)

However, advantageously, the step of washing can also be implemented in a chamber chosen from a storage tray, a de-salting capacity, a tank, in particular a separator tank of the water/oil type. The washing can advantageously be carried out in an already-existing chamber of a treatment unit.

The product coming from the step of washing is recovered usually by a water/oil separation technique, for example by centrifugation and/or settling and/or with the application of an electric field. When the separation is carried out by settling, it can be carried out in the same chamber as the one that was used for washing.

The recovered product, in particular directly, at the end of this step of washing can either constitute a refined composition, in particular a refined oil that can then be subjected directly to a step of hydrotreatment, or a partially refined oil, which can then be directly subjected either to a step of removing compounds that are hydratable at a pH other than a neutral pH then to a step of removing non-hydratable compounds, or only to a step of removing non-hydratable compounds, before a later step such as a hydrotreatment.

Step of Removing Compounds that are Hydratable at a pH Other than a Neutral pH (at a Basic pH or Acid pH)

Typically, this step makes it possible to remove the hydratable compounds, in particular of phosphorus, of nitrogen and of metals, and a portion of the chlorine, that were not removed during the step of washing with neutral pH water.

This step of removing can comprise one or more treatments chosen for example from a degumming treatment in an acid or basic medium and a treatment by cavitation, with each treatment being followed by a step of separating the oily and aqueous phases, the recovered oily phase having a reduced content in compounds that are hydratable at a pH other than a neutral pH. This separation can be carried by means of any usual separating technique.

It is in particular possible to carry out several degumming treatments in acidic and basic mediums in order to remove the compounds that are hydratable at an acid pH and basic pH respectively.

The degumming treatment can be a water degumming treatment during which the oil to be treated is typically heated to 60-70° C., water to which has been added a basic compound (for example NaOH) or acid compound (for example citric or phosphoric acid) is added and mixed for about 30 minutes, then the hydrated gums are separated by centrifugation and the degummed oil is vacuum dried. This method often entails adding live steam to the product to be treated for a short period. The suitable quantity of water normally represents about 75% by weight of the phosphatide content of the oil to be treated. An insufficient quantity of water produces dark viscous gums, while an excessive quantity of water results in excessive losses of the oil by hydrolysis. A water degummed oil generally still contains phosphatides (between 50 and 200 ppm by weight).

The degumming treatment can be a treatment with acid during which the oil to be treated is typically heated to 60-70° C., and a water-acid mixture is added and mixed for about 30 minutes. Typically phosphoric acid or citric acid is used.

The degumming treatment can be an enzymatic degumming treatment wherein an enzyme, for example phospholipase A1, the most recent degumming enzyme, transforms the phospholipids into lysophospholipids and into free fatty acids. This process comprises three major steps:
(1) adjustment of the pH with a buffer;
(2) enzymatic reaction in the retention basins; and
(3) separating of the sludge and oil.

The oil to be degummed enzymatically in this way can be crude or degummed beforehand with water.

The lipid manual (The lipid handbook, edited by Frank D. Gunstone, John L. Harwood, Albert J. Dijkstra. 3rd ed.) describes many variants and details of the degumming treatments.

The treatment can be a treatment by cavitation, and in particular by hydrodynamic cavitation, of the oil to be treated in the presence of water in conditions that are effective for generating cavitation characteristics and for transferring at least a portion of the impurities contained in the oil in an aqueous phase.

Cavitation is the phenomenon of the formation of steam bubbles in a liquid flowing in regions where the pressure of the liquid is less than its steam pressure at the temperature considered.

Cavitation is a nucleation, growth and implosion (collapse) phenomenon of cavities filled with steam or gas, which can be obtained by passing ultrasound (acoustic cavitation), by a laser, by injecting steam into a cold fluid or by modifications to the flow and pressure (hydrodynamic cavitation).

Hydrodynamic cavitation can be generated by passing the mixture to be treated through one or more cavitation devices.

The hydrodynamic cavitation method can therefore comprise the following steps:
the pumping of the oil to be treated through a cavitation device,
the generating of cavitation characteristics to remove the impurities.

Suitable cavitation devices that can be used are for example disclosed in WO201098783A1, U.S. Pat. No. 8,911,808B2, U.S. Pat. No. 7,762,715B2, U.S. Pat. No. 8,042,989B2.

For example, a suitable cavitation device comprises a flow path through which the fluid is pumped, such as the one disclosed in U.S. Pat. No. 8,911,808B2, wherein a predetermined pump pressure is applied preferably in the range of 340 kPa-34 MPA.

In the treatment via hydrodynamic cavitation, the phosphatides are hydrated in gums, which are insoluble in oil and can easily be separated in the form of sludge forming an aqueous phase, for example by settling, filtering or centrifugal action.

In an embodiment, the treatment by cavitation, in particular by hydrodynamic cavitation can be carried out in the presence of a degumming agent. The latter can be chosen from water, steam, acids, complexing agents and mixtures thereof.

The acids are for example strong acids, in particular inorganic acids, such as phosphoric acid, sulfuric acid.

The complexing agents or for example weak organic acids (or their corresponding anhydrides) such as acetic acid, citric acid, oxalic acid, tartaric acid, malic acid, maleic acid, fumaric acid, aspartic amino acid, ethylenediamine-tetra-acetic acid (EDTA).

Preferably, the degumming agent comprises water, steam, phosphoric acid, acetic acid, citric acid, oxalic acid, tartaric acid, malic acid, fumaric acid, aspartic amino acid, ethylenediamine-tetra-acetic acid, a base, salts, chelating agents, crown ethers or maleic anhydride.

The treatment by cavitation can be carried out at temperatures close to ambient temperature or lower than the latter, for example at 15-25° C. However hydrodynamic cavitation can be carried out between 1° and 90° C., preferably between 25 and 75° C. and more preferably between 3° and 60° C.

It is possible for example to proceed as described in document WO2019229035A1.

The product coming from this step of removal can be separated by one or more of the following known techniques: sedimentation, centrifugation, filtration, distillation, extraction or washing, preferably sedimentation, centrifugation, filtration. The product recovered after separating the aqueous water is an oily phase which constitutes a composition or oil that is partially refined that can then be subjected to a step of removal of non-hydratable compounds.

### Step of Removing Non-Hydratable Compounds

Typically, this step makes it possible to remove the non-hydratable compounds, in particular of phosphorus, of nitrogen, such as the non-hydratable phosphatides (calcium and magnesium salts of phosphatidic acid and of phosphatidyl ethanolamine), as well as of other elements such as sulfur, and/or a portion of the residual organic chlorine, that were not eliminated during the step of washing with neutral pH water and the optional step of removing compounds that are hydratable at a pH other than a neutral pH.

This step typically comprises one or more treatments chosen from a bleaching treatment wherein the product to be treated is put into contact with an absorbent, a treatment wherein the product to be treated is put into contact with an ion-exchanging resin, a mild acid wash, a treatment using guard beds, a filtration, an extraction by solvent, each treatment being followed by a step of separating the oily and aqueous phases, the recovered oily phase forming a composition having a reduced content in non-hydratable compounds. This separation can be carried out by means of any separating technique.

Bleaching is a well-known technique, generally used to discolor and purify chemically or physically refined oils. It generally provides the removal of soaps, residual phosphatides, trace metals and certain oxidation products. It catalyzes the removal of carotene and the absorbent also catalyzes the decomposition of peroxides. Another function is the removal of peroxides and secondary oxidation products.

This treatment consists of putting the product to be treated in contact with an absorbent, such as absorbent clays, synthetic amorphous silica and activated carbon. Optionally, prior to this putting into contact, the product to be treated can be mixed with an acid in order to decompose the metal ion/phosphatide complexes.

The key parameters of the bleaching treatment are the type and dosage of the absorbent, the temperature, the time, the humidity and the filtration, as indicated in the Lipids handbook, Edited by Frank D. Gunstone, John L. Harwood, Albert J. Dijkstra. 3rd ed., chapter 3.7); or in the “practical guide to vegetable oil processing”, 2^{nd} edition, Monoj. K. Gupta.

Another possible treatment is a treatment by ion-exchanging resin. This treatment consists of putting the product to be treated in contact with an ion-exchanging resin in a pre-treatment zone, in pre-treatment conditions. The ion-exchanging resin is for example an acid ion-exchanging resin such as Amberlyst™-15 and can be used as a bed in a reactor through which the product to be treated flows, either upstream, or downstream.

Another possible treatment is a washing with mild acid. This treatment is carried out by putting the product to be treated in contact with an acid such as sulfuric, nitric, phosphoric or hydrochloric acid in a reactor. The acid in the product to be treated can be put into contact in a discontinuous or continuous process. The putting into contact is done with a diluted acid solution, generally at ambient temperature and at atmospheric pressure. If the putting into contact is carried out continuously, it is generally done at countercurrent.

Another possible treatment is the use of guard beds, well known in the art. These can be guard beds containing alumina, with or without demetallization catalysts such as nickel, cobalt and/or molybdenum.

Filtration and extraction techniques by solvent are other choices that can be used.

The recovered product, in particular directly, at the end of this step of removing non-hydratable compounds constitutes a refined composition or oil that can then be subjected to a step of converting into a fuel of natural origin, preferably by hydrotreatment.

### Step of Hydrotreatment

The method for decreasing the heteroatom content of a renewable oil composition according to the invention can further comprise (E3) a hydrotreatment step of the product directly at the end of step (E2) or (E′2).

The composition treated in this hydrotreatment step is thus a refined composition, preferably directly coming from one of the treatments (i) to (iv).

This refined composition typically has at least one of the following characteristics, preferably all of them:
A phosphorus content less than 200 ppm, preferably less than 100 ppm or 50 ppm, more preferably less than 3 ppm,
A nitrogen content less than 200 ppm, preferably less than 100 ppm, more preferably less than 50 ppm,
A chlorine content less than 50 ppm, preferably less than 20 ppm, more preferably less than 5 ppm
A metal content less than 50 ppm, preferably less than 10 ppm

In an embodiment, the refined composition can be hydrotreated mixed with one or more mineral hydrocarbon fractions. The mineral hydrocarbon fraction or fractions can be of the naphtha, kerosene or diesel type. This mineral hydrocarbon fraction can be added in quantities ranging from 1 to 98% by mass or from 1 to 95% by mass or in any interval defined by two of these limits. This mineral hydrocarbon fraction, which contains generally organic sulfur compounds, will provide the sulfur required to maintain the catalytic activity of the catalyst containing cobalt, nickel, tungsten and molybdenum. At the same time, the mineral hydrocarbon fraction is also at least partially desulfurized.

This hydrotreatment step is typically carried out in the presence of dihydrogen and of at least one catalyst to transform the fatty acid esters and the free fatty acids, contained in the product directly coming from one of the treatments (i) to (iv) into linear or substantially linear paraffins. The hydrotreatment can be carried out between 10° and 550° C. in the presence of dihydrogen at pressures ranging from 0.01 to 10 MPa. The ratio between the dihydrogen and the supply feedstock can be from 100 to 2000 NI/I.

This hydrotreatment can comprise one or more of the steps chosen from hydrodeoxygenation, decarboxylation and decarbonylation.

The hydrodeoxygenation is preferably done in continuous fixed bed reactors, reactors with a continuously stirred reservoir or reactors of the slurry type containing a solid catalyst which can be chosen from the oxides or sulfurs of Ni, Mo, W, Co or mixtures such as NiW, NiMo, CoMo, NiCoW, NiCoMo, NiMoW and CoMoW as catalytic phase, preferably supported on carbon, alumina, silica, titanium oxide or zirconia.

The hydrodeoxygenation can be carried out at a temperature from 200 to 500° C., preferably from 220 to 400° C., under pressure of 1 MPa to 10 MPa (10 to 100 bar), for example 6 MPa, and with a dihydrogen/oil ratio from 100 to 2000, but preferably from 350 to 1500, for example 800 NI H₂/I of oil.

The decarboxylation and/or decarbonylation is done preferably in the presence of a solid catalyst in reactors with a discontinuous type tank, continuous reactors with a fixed bed, reactors with a continuously stirred tank or sludge reactors. The decarboxylation and/or decarbonylation can be done directly with glycerides, any esters or with free fatty acids.

The catalyst can be chosen from:
the oxides or sulfides of Ni, Mo, W, Co, NiW, NiMo, CoMo, NiCoW, NiCoMo, NiMoW and CoMoW as catalytic phase, preferably supported on carbon, alumina, silica, titanium oxide or zirconia, or
metals or mixtures of alloys of group 10 (Ni, Pt and Pd) and of group 11 (Cu and Ag) supported by carbon, magnesia, zinc oxide, spinels (Mg₂Al₂O₄, ZnAl₂O₄), perovskites (BaTiO₃, ZnTiO₃), calcium silicates (such as xonotlite), alumina, silica or silicas-aluminas or mixtures of the latter.

For optimum performance and continuous stable operation, it is preferable that the active metal component of the catalyst in the case of Ni, Mo, W, Co or mixtures, be in the form of sulfides. It is therefore preferable that traces of decomposable sulfur compounds (thermally or catalytically) be present or added voluntarily to the feedstock so as to maintain the metal sulfide in its state of sulfide. As an example, these sulfur compounds can be H₂S, COS, CS₂, mercaptans (for example, methylsulfide), thioethers (for example, dimethylsulfide), disulfides (for example, dimethyl disulfide), thiopheneic and tetrahydrothiophenic compounds.

The decarboxylation and/or decarbonylation can also be carried out on basic oxides, such as, alkali metal oxides, alkaline earth oxides, lanthanide oxides, zinc oxide, spinels (Mg₂Al₂O₄, ZnAl₂O₄), perovskites (BaTiO₃, ZnTiO₃), calcium silicates (such as xonotlite), either in bulk, or dispersed on neutral or basic supports, on basic zeolites (such as alkaline or alkali earth zeolites with a low silica/alumina content obtained by exchange or impregnation).

Although the decarboxylation and/or decarbonylation reaction does not require dihydrogen, it is preferable that the decarboxylation and/or decarbonylation be carried out in the presence of dihydrogen which will stabilize the catalytic activity by removing the highly absorbed unsaturated species (for example when the decarbonylation is the predominant reaction path) from the surface of the catalyst by hydrogen addition reactions. The presence of the dihydrogen can also hydrogenate the double bonds present in the acyl portion of the fatty acid in order to obtain paraffin reaction products from the decarboxylation process.

The step of decarboxylation and/or decarbonylation can be carried out between 10° and 550° C. in the presence of dihydrogen at pressures ranging from 0.01 to 10 MPa. The ratio between the dihydrogen and the supply feedstock can be from 100 to 2000 NI/I.

### System for Determining the Treatment for the Removal of Heteroatoms

The invention also has for object a system for determining the treatment for the removal of heteroatoms to be applied to a composition of natural origin to reach a target content in at least one heteroatom, configured, in particular programmed, to implement the steps (a) to (c) of the method for determining according to the invention.

The system for determining typically comprises one or more processors, for example a microprocessor, a microcontroller or other. It can be configured to receive the total content of said composition in at least one heteroatom chosen from phosphorus and nitrogen and the total content of said composition in hydratable compounds of the at least one heteroatom.

It typically comprises output or input/output interfaces. This can be wireless communication interfaces (Bluetooth, WIFI or other) or connectors (network port, USB port, serial port, Firewire® port, SCSI port or other). These input and/or output interfaces can form means of communication, optionally bidirectional, between the system for determining and for example a user interface, allowing in particular the system to receive the contents relative to the composition.

The system for determining can also comprise means of storage which can be a random-access memory (RAM), an electrical erasable programmable read-only memory (EEPROM), a flash memory, an external memory or other. These means of storage can, among others, store the data received, the measured values, the calculated values, a database and/or a model, and one or more computer programs.

The system can thus comprise at least one processor configured to:
(a) receive the total content of said composition in at least one heteroatom chosen from phosphorus and nitrogen, receive the total content of said composition in hydratable compounds of the at least one heteroatom and determine as a representative parameter of the quality of the composition, the ratio of the total content in the at least one heteroatom present in the neutral pH hydratable compounds of this heteroatom identified over the total content of the composition in this heteroatom, this ratio being determined for phosphorus, for nitrogen or for each one of these two heteroatoms,
(b) estimate a residual content in at least one heteroatom chosen from phosphorus and nitrogen, using the representative parameter, this residual content corresponding to the content in the at least one heteroatom after removal of the hydratable compounds at pH=7 containing this heteroatom,
(c) determine a treatment to be applied to said composition to reach a target content in the at least one heteroatom at the output of the treatment, this treatment been chosen from:
(i) a washing with neutral pH water,
(ii) a washing with neutral pH water followed by a step of removing non-hydratable compounds,
(iii) a washing with neutral pH water followed by a step of removing compounds hydratable at a pH other than a neutral pH,
(iv) a washing with neutral pH water followed by a step of removing compounds hydratable at a pH other than a neutral pH then a step of removing non-hydratable compounds, the at least one processor being configured to:
compare said residual content to the target content and
choose the treatment (i) if the residual content is less than or equal to the target content,
otherwise, use a database or a model configured to supply, an estimate of the content in the at least one heteroatom at the output of each treatment (ii) to (iv) according to a content in this heteroatom at the input of the treatment equal to the residual content, and choose the treatment for which the estimated content at the output is less than or equal to the target content, and optionally for which the number of treatment steps is the lowest.

The at least one processor can further be configured to:
(c) if none of the estimated contents at the output is less than or equal to the target content, reiterate the steps (a) to (c) after dilution of said composition of natural origin with another composition of natural origin, optionally a composition having a representative parameter of the quality of the composition that is higher than that of said composition,
and/or
(c) choose a treatment and the operating conditions of this treatment for which the estimated content at the output is less than or equal to the target content, and optionally for which the number of treatment steps is the lowest and/or for which the quantity of reagent used is the lowest, the database or the model being configured to supply an estimate, for each treatment (ii) to (iv), of the contents in the at least one heteroatom at the output of said treatment, obtained for different operating conditions of the treatments (i) to (iv)

In all cases, the at least one processor can receive data via one or more input, or input and output, interfaces, in particular of the aforementioned type, and can store a database or a model in means of storage, in particular of the aforementioned type.

### FIGURES

Other particularities and advantages of the invention shall appear when reading the description given hereinafter of a particular embodiment of the invention, given for the purposes of information but in a non-limiting manner, in reference to the annexed drawings wherein:

FIG. 1 diagrammatically shows an example embodiment of the method of treating according to the invention.

FIG. 2 diagrammatically shows a washing chamber.

FIG. 3 is a table showing the chemical formulas of phospholipids in water according to the pH, the R group represents a diglyceride.

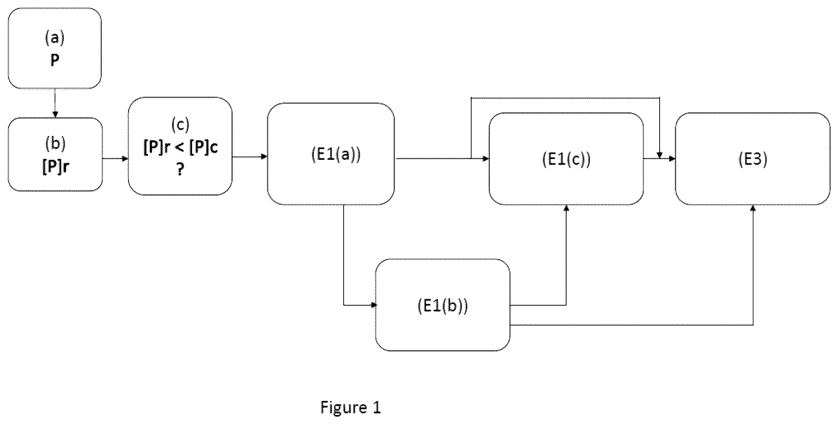

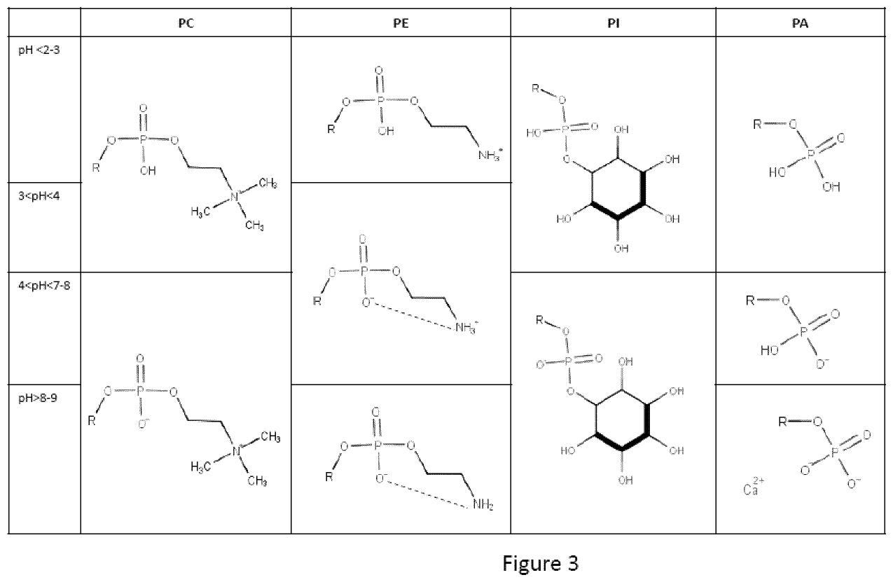

FIG. 1 diagrammatically shows a method for decreasing the heteroatom content of a composition of natural origin H.

The composition of natural origin H to be treated is first subjected to the step (a) of determining a parameter Q representative of its quality, here the parameter relating to the phosphorus content.

This parameter Q can be written (equation 1):

$$[ Math 1 ]$$

$$Q = \frac{\sum [ PC , Salts P ; PI ]}{[ P ]} \qquad ( equation 1 )$$

where
ΣPC, Salts P; PI] represents the total content of the composition H in phosphorus present in the compounds of the phosphorus hydratable at neutral pH, namely hydratable phosphorus salts, PC and PI, and [P] represents the total content in phosphorus (elemental) of the composition H.

During the step (b) the residual content in phosphorus is estimated, noted as [P]r, from the parameter Q. This residual content corresponds to the total content in phosphorus from which is subtracted the phosphorus content present in the compounds of the phosphorus hydratable at neutral pH and can therefore be calculated in the following way (equation 2):

$$[ Math 2 ]$$

$$[ P ] r = [ P ] \times ( 1 - Q ) \qquad ( equation 2 )$$

During the step (c), this residual content [P]r is then compared with the desired target content (noted as [P]c) and one of the treatments (i) to (iv) is chosen such as described hereinabove, and the composition is subjected to the treatment retained which makes it possible to obtain a refined composition.

Thus, such as shown in FIG. 1:
if [P]r<[P]c, only the step of washing with neutral pH water E1(a) is implemented before the hydrotreatment step E3,
If [P]r>[P]c, it is possible to implement, before the hydrotreatment step E3, the succession of steps E1(a) and E1(c) (removal of non-hydratable compounds), the succession of steps E1(a), E1(b) (removal of compounds that are hydratable at a pH other than a neutral pH), or the succession of steps E1(a), E1(b), E1(c).

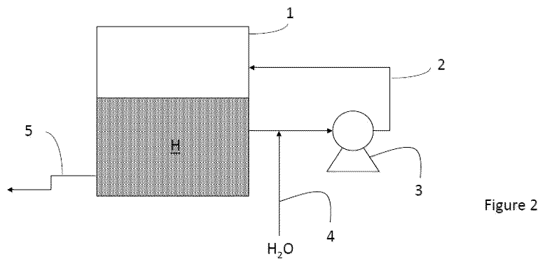

FIG. 2 shows a chamber 1, for example a storage tray or a de-salting capacity containing the composition H. This chamber 1 is equipped with a recirculation circuit 2 provided with a pump 3. Upstream of the pump 3 with respect to the direction of recirculation of the recirculation circuit 2, a duct 4 makes it possible to inject water into the circuit 2. A tapping duct 5 located at the bottom of the chamber 1 makes it possible to recover the oil after washing with water.

### EXAMPLES

### Example 1: Analysis of the Chosphorus Content of Different Compositions of Natural Origin

Table 1 shows the analysis results of the organic phase and of the aqueous phase recovered after a liquid/liquid extraction of different compositions with an aqueous solution at pH=7 containing EDTA.

The measurements of the ³¹P NMR in the organic phase were carried out in deuterated chloroform (CDCl₃) and those carried out in the aqueous phase were carried out in a mixture of deuterated water (D₂O) and methyl alcohol (MeOH), for example according to the protocol described hereinafter.

A precise quantity of the renewable oil sample is taken in a vial to which is added a precise quantity of a phosphorus standard soluble in the organic phase and of a phosphorus standard soluble in the aqueous phase. This sample is then solubilized in deuterated chloroform. A solution of 0.2 M of EDTA at pH7 in MeOH/D₂O is then added to maximize the analytical resolution, by inspiration from articles in the literature: T. Glonek, M. Lunde, M. Mudgett, T. C. Myers (1971) Studies of Biological Polyphosphate Through the Use of Phosphorus-31 Nuclear Magnetic Resonance. In: Archives of Biochemistry and Biophysics, vol. 142, p. 508-513; T. O. Hendersen, T. Glonek, T. C. Myers (1974) Phosphorus-31 Nuclear Magnetic Resonance Spectroscopy of Phospholipids. In: Biochemistry, vol. 13, n^{o}3, p. 623-628. The aqueous and organic phases are then separated by settling for one night and analyzed separately by ³¹p NMR. The identification of the species is done thanks to their chemical shift by comparison with standards of equivalent structure and their quantification is done by the ratio of the intensity of their ³¹p signal over the intensity of the standard soluble in the same phase initially introduced into the sample.

The composition C1 is a poultry fat, the composition C2 is a mixture of fats coming from several animals, the composition C3 is pig fat.

TABLE 1

| Composition | | Unit | C1 | C2 | C3 |
|---|---|---|---|---|---|
| Elemental analysis of the | Content in P | Ppm (³¹P | 500 | 132 | 751 |
| composition by XRF | | element) | | | |
| Analysis by ³¹P NMR of the | Content in P present in PC | Ppm (³¹P | 87 | 32 | 42 |
| organic phase | | element) | | | |
| | Content in P present in PI | Ppm (³¹P | ND | 8 | 8 |
| | | element) | | | |
| | Content in P present in the | Ppm (³¹P | 233 | 35 | 32 |
| | other compounds containing | element) | | | |
| | phosphorus | | | | |
| | Total content in P present in | Ppm (³¹P | 320 | 75 | 82 |
| | the organic compounds | element) | | | |
| | containing phosphorus | | | | |
| Analysis by ³¹P NMR of the | Content in P present in the | Ppm (³¹P | 165 | 35 | 421 |
| aqueous phase | phosphates | element) | | | |
| | Content in P present in the | Ppm (³¹P | ND | ND | 20 |
| | other phosphorus compounds | element) | | | |
| Total content in phosphorus | Content in P | Ppm (³¹P | 485 | 110 | 524 |
| of the composition obtained | | element) | | | |
| by NMR analysis | | | | | |
| Total content in P present in | Content in P | Ppm (³¹P | 252 | 75 | 491 |
| the compounds of the P | | element) | | | |
| hydratable at pH 7 | | | | | |
| Parameter Q | | | 0.50 | 0.57 | 0.65 |

The data in table 1 makes it possible to calculate the total content of the phosphorus hydratable at pH 7 (corresponding to the sum of the contents in P coming from PC and PI and from the aqueous phase—the added values are in bold in the table) and the parameter Q representative of the quality of each one of the compositions calculated by dividing the total content in P present in the compounds of the P hydratable at pH 7 by the total content P of the composition measured by XRF.

### Example 2—Washing with pH Neutral Water

The composition C3 of the example 1 was subjected to a washing with neutral pH water with water/fat ratios of 4, 8 and 12.

The water and the fat were separated by centrifugation at 4800 g before measurement of the phosphorus content of the oily phase.

Table 2 shows the phosphorus contents of the oily phase measured by XRF for the various ratios tested.

TABLE 2

| | | | Phosphorus |
|---|---|---|---|
| Treatment | Phosphorus | Water/fat | content of the |
| composition C3 | content (ppm) | ratio (% m) | oily phase (ppm) |
| No treatment | 751 | — | — |
| Washing with water | | 4 | 7.2 |
| | | 8 | 7.8 |
| | | 12 | 6.6 |

Note that a substantial reduction in phosphorus is observed with washing with water with 4% m of water. Thus, for a fat composition having a parameter representative of the quality of the composition C3 of 0.65, a step of washing with water makes it possible to remove almost all of the phosphorus present in the composition, which could then possibly be directly treated in a hydrotreatment unit so as to produce a fuel according to the specification in phosphorus of the feedstocks entering into this hydrotreatment unit.

1. Method for determining a parameter representative of the quality of a composition of natural origin comprising heteroatoms and lipids chosen from phenolic lipids, fatty acids, triglycerides, diglycerides, monoglycerides, phospholipids, fatty acid esters and/or a mixture of two or more of these compounds, the method comprising:
(A) determining the total content of said composition in at least one heteroatom chosen from phosphorus and nitrogen,
(B) identifying compounds of the at least one heteroatom which are hydratable at neutral pH and determining the total content of said composition in the at least one heteroatom present in the identified neutral pH hydratable compounds of this heteroatom,
(C) determining as a parameter representative of the quality of the composition of the ratio of the total content in the at least one heteroatom present in the identified neutral pH hydratable compounds of this heteroatom over the total content of the composition in this heteroatom, this ratio being determined for phosphorus, for nitrogen or for each one of these two heteroatoms.

2. Method for determining according to claim 1, wherein the step (B) is carried out using an analysis technique chosen from NMR, mass spectroscopy, ion chromatography, liquid chromatography coupled with mass spectrometry and high-performance thin-layer chromatography.

3. Method for determining according to claim 1, wherein said composition of natural origin contains one or more oils chosen from a vegetable oil, an animal oil or fat, a used oil, an oil produced by microorganisms, esters resulting from the transesterification of fatty acid esters contained in one or more of these oils, as well as mixtures thereof.

4. Method for determining the treatment for the removal of heteroatoms to be applied to a composition of natural origin comprising heteroatoms and lipids chosen from phenolic lipids, fatty acids, triglycerides, diglycerides, monoglycerides, phospholipids, fatty acid esters and/or a mixture of two or more of these compounds to reach a target content for at least one heteroatom, the method comprising:
(a) a step of determining at least one parameter representative of the quality of the composition by means of the method for determining according to claim 1,
(b) a step of estimating a residual content in at least one heteroatom chosen from phosphorus and nitrogen, from the representative parameter, this residual content corresponding to the content in the at least one heteroatom after removal of the compounds hydratable at pH=7 containing this heteroatom,
(c) step of determining a treatment to be applied to said composition to reach a target content in the at least one heteroatom at the output of the treatment, said treatment being chosen from:
(i) a washing with neutral pH water,
(ii) a washing with neutral pH water followed by a step of removing non-hydratable compounds,
(iii) a washing with neutral pH water followed by a step of removing compounds hydratable at a pH other than a neutral pH,
(iv) a washing with neutral pH water followed by a step of removing compounds hydratable at a pH other than a neutral pH then a step of removing non-hydratable compounds,
wherein
said residual content is compared to the target content and
The treatment (i) is chosen if the residual content is less than or equal to the target content,
otherwise, a database or a model is used to configure to supply, an estimate of the content in the at least one heteroatom at the output of each treatment (ii) to (iv) according to a content in this heteroatom at the input of the treatment equal to the residual content, and choose the treatment for which the estimated content at the output is less than or equal to the target content, and optionally for which the number of treatment steps is the lowest.

5. Method for determining according to claim 4, wherein, during the step (c), if none of the estimated contents at the output is less than or equal to the target content, steps (a) to (c) are reiterated by diluting said composition of natural origin with another composition of natural origin, optionally a composition having a parameter representative of the quality of the composition greater than that of said composition.

6. Method for determining according to claim 4, wherein, the database or the model is configured to supply an estimate, for each treatment (ii) to (iv), of the contents in the at least one heteroatom at the output of said treatment, obtained for different operating conditions of the treatments (i) and (iv) and during the step (c), the treatment and the operating conditions of this treatment are chosen for which the estimated content at the output is less than or equal to the target content, and optionally for which the number of treatment steps is the lowest and/or for which the quantity of reagent used is the lowest.

7. Method for determining according to claim 4, wherein said composition of natural origin contains one or more oils chosen from a vegetable oil, an animal oil or fat, a used oil, an oil produced by microorganisms, esters resulting from the transesterification of fatty acid esters contained in one or more of these oils, as well as mixtures thereof.

8. Method for decreasing the heteroatom content of a composition of natural origin comprising heteroatoms and lipids chosen from phenolic lipids, fatty acids, triglycerides, diglycerides, monoglycerides, phospholipids, fatty acid esters and/or a mixture of two or more of these compounds, the method comprising:
(E1) a step of determining the treatment for the removal of heteroatoms to be applied to the composition of natural origin to reach a target content in at least one heteroatom, by means of the method for determining according to claim 4, and
(E2) a step during which said composition of natural origin is subjected to the treatment determined in step (E1).

9. Method for decreasing the heteroatom content of a composition of natural origin comprising heteroatoms and lipids chosen from phenolic lipids, fatty acids, triglycerides, diglycerides, monoglycerides, phospholipids, fatty acid esters and/or a mixture of two or more of these compounds, the method comprising:
(E′2) A step for the removal of heteroatoms during which the composition of natural origin is subjected to a treatment chosen from:
(i) a washing with neutral pH water,
(ii) a washing with neutral pH water followed by a step of removing non-hydratable compounds,
(iii) a washing with neutral pH water followed by a step of removing compounds hydratable at a pH other than a neutral pH,
(iv) a washing with neutral pH water followed by a step of removing compounds hydratable at a pH other than a neutral pH then a step of removing non-hydratable compounds,
and wherein, the composition of natural origin has a parameter representative of the quality of the composition, determined beforehand by means of the method for determining according to claim 1, which is greater than or equal to a threshold beyond which the effectiveness of the step of removing is sufficient to reduce the heteroatom content of said composition to a target content or less than a target content.

10. Method for decreasing according to claim 8, further comprising (E3) a step of hydrotreatment of the product directly coming from the step (E2) or (E′2).

11. Method for decreasing according to claim 8, wherein the composition of natural origin contains one or more oils chosen from a vegetable oil, an animal oil or fat, a used oil, an oil produced by microorganisms, esters resulting from the transesterification of fatty acid esters contained in one or more of these oils, as well as mixtures thereof.

12. Method for decreasing according to claim 8, wherein, during the step (E2) or (E′2), with neutral pH water of each one of the treatments (i) to (iv) is implemented with a water/composition mass ratio of 1:99 to 10:90.

13. Method for decreasing according to claim 8, wherein, during the step (E2) or (E′2), the washing with neutral pH water of each one of the treatments (i) to (iv) is implemented in a chamber chosen from a storage tray, a de-salting capacity, a tank.

14. System for determining the treatment for the removal of heteroatoms to be applied to a composition of natural origin to reach a target content in at least one heteroatom, configured to implement the steps (a) to (c) of the method for determining according to claim 4, the system comprising at least one processor configured to:
(a) receive the total content of said composition in at least one heteroatom chosen from phosphorus and nitrogen, the total content of said composition in hydratable compounds of the at least one heteroatom and to determine as a representative parameter of the quality of the composition, the ratio of the total content in the at least one heteroatom present in the identified neutral pH hydratable compounds of this heteroatom over the total content of the composition in this heteroatom, this ratio being determined for phosphorus, for nitrogen or for each one of these two heteroatoms,
(b) estimate a residual content in at least one heteroatom chosen from phosphorus and nitrogen, using the representative parameter, this residual content corresponding to the content in the at least one heteroatom after removal of the hydratable compounds at pH=7 containing this heteroatom,
(c) determine a treatment to be applied to said composition to reach a target content in the at least one heteroatom at the output of the treatment, this treatment being chosen from:
(i) a washing with neutral pH water,
(ii) a washing with neutral pH water followed by a step of removing non-hydratable compounds,
(iii) a washing with neutral pH water followed by a step of removing compounds hydratable at a pH other than a neutral pH,
(iv) a washing with neutral pH water followed by a step of removing compounds hydratable at a pH other than a neutral pH then a step of removing non-hydratable compounds,
the at least one processor being configured to:
compare said residual content to the target content and
choose the treatment (i) if the residual content is less than or equal to the target content, otherwise, use a database or a model configure to supply, an estimate of the content in the at least one heteroatom at the output of each treatment (ii) to (iv) according to a content in this heteroatom at the input of the treatment equal to the residual content, and choose the treatment for which the estimated content at the output is less than or equal to the target content, and optionally for which the number of treatment steps is the lowest.

15. System for determining according to claim 14, wherein the at least one processor is further configured to:
(c) if none of the estimated contents at the output is less than or equal to the target content, reiterate the steps (a) to (c) after dilution of said composition of natural origin with another composition of natural origin, optionally a composition having a representative parameter of the quality of the composition that is higher than that of said composition,
and/or
(c) choose a treatment and the operating conditions of this treatment for which the estimated content at the output is less than or equal to the target content, and optionally for which the number of treatment steps is the lowest and/or for which the quantity of reagent used is the lowest, the database or the model being configured to supply an estimate, for each treatment (ii) to (iv), contents in the at least one heteroatom at the output of said treatment, obtained for different operating conditions of the treatments (i) to (iv).
